(19) United States
(12) Patent Application Publication
(10) Pub. No.: US 2023/0079047 A1
(43) Pub. Date: Mar. 16, 2023

(54) GENERATOR AND METHOD FOR PRODUCING A TREATMENT VOLTAGE

(71) Applicant: Erbe Elektromedizin GmbH, Tuebingen (DE)

(72) Inventor: Konstantin Dornhof, Horb am Neckar (DE)

(21) Appl. No.: 17/942,251

(22) Filed: Sep. 12, 2022

(30) Foreign Application Priority Data
Sep. 14, 2021 (EP) 21196607.2-1113

(51) Int. Cl. A61B 18/12

(52) U.S. Cl. CPC A61B 18/1206; A61B 2018/00827

(57) ABSTRACT

A generator includes a number of impulse generators that are individually controlled by means of a control device in a timely flexible manner. The RF voltage required for supply of a surgical instrument is thus composed of individual impulses. The same applies for the current flowing at the electrode of the instrument. Due to omitting resonance effects in the impulse generators and omitting of energy storage in a system that is able to oscillate (system of second order), the user has an increased degree of control of the wave forms of the voltage supplied to the instrument and the current flowing to the instrument.

### RELATED APPLICATION(S)

This application claims the benefit of European Patent Application No. 21196607.2-1113, filed Sep. 14, 2021, the contents of which are incorporated herein by reference as if fully rewritten herein.

### TECHNICAL FIELD

The invention refers to a generator for supply of medical instruments with a treatment voltage and a treatment current as well as a method for producing such a treatment voltage and a treatment current.

### BACKGROUND

Electrosurgical instruments, probes and the like for use in the electrical surgery are typically supplied with radio frequency alternating current. The frequency of this alternating current or the applied alternating voltage is typically over 100 kHz in order to avoid neuromuscular irritations. The power of such generators is typically remarkably over 1 W and can reach multiple 100 W.

For producing electrosurgical voltages in the range of multiple 100 kHz externally controlled generators are typically used, as for example apparent from DE 10 2008 039 884 A1. Such a generator comprises at least one oscillation circuit that is made to oscillate by means of an active transistor circuit and from which electrosurgical energy is withdrawn in a transformer-type manner. Different concepts are known in order to modulate the generator voltage for achieving different tissue effects.

The variation of the oscillation in an oscillation circuit is subject to time restrictions, whereby also constraints are put on the type of modulation.

It is one object of the invention to provide a generator that allows extended creation possibilities with regard to the produced oscillation and voltage shapes.

### SUMMARY

This object is solved by means of a generator as well as a method as disclosed herein.

The generator according to the invention comprises a number of impulse generators that respectively have a control input and an impulse generator output. Preferably each impulse generator is configured such that it provides an output impulse upon receipt of a control impulse at its control input. Preferably the outputs of the impulse generators are connected with the output of the generator at which the output impulses arrive, which are produced by the impulse generators. A control device is connected with the control inputs of the impulse generators and coordinates their impulse output. In this manner impulse sequences having impulses of equal or different strength as well as periodically repeating impulses or also impulse sequences with different, changing time intervals can be produced. Thus, a large number of possible voltage and current shapes are possible at the output of the generator that cannot be created or can only be created with disproportionate efforts by means of a parallel oscillation circuit. Impulse sequences having symmetric impulse shapes, particularly however also impulse sequences with asymmetric impulse shapes can be created.

The concept according to the invention distributes the output power of the generator onto multiple impulse generators that contribute in this manner individually only for a power portion contribution respectively to the total power that is output by the generator. This minimizes the stress of the individual components in the impulse generators, particularly their power switches and reduces the cooling power need. In doing so, the power switches can be configured as integrated components, e.g. as transistor arrays.

Preferably the impulse generators are not systems that are able to oscillate on their own, i.e. they preferably do not comprise any resonance components, resonance circuits or the like that are connected with the impulse generator output (i.e. the output circuit is described with a differential equation, the order of which is less than 2, at least with reference to solutions with frequencies that are within the magnitude of the output frequency). For this reason, resonance effects fail to appear, which is why the impulse generators can output impulses in a controlled manner without post-pulse oscillation. Upon output of their impulses the impulse generators respectively output the entire stored power as output impulse.

This provides an improved control for the generator over the voltage shapes and the power output to the instrument and thus to the biological tissue.

Preferably the impulse generators are configured identically compared with each other. They are thus identical in construction and create output impulses of equal magnitude. Due to the timing of the impulse output of the individual impulse generators, the output impulse sequence can be designed. For example, impulse generators can fire (deliver output impulses) concurrently, such that the output impulses of the impulse generators are added at the generator output. Also impulse sequences can be created that contain individual impulses provided in a predefined time scheme. It is in addition possible to provide impulse generators that deliver output impulses having different magnitudes, e.g. in order to provide output impulses at the output of the generator having different magnitudes due to different combinations of output impulses of individual impulse generators.

The control device is preferably configured to output control impulses in selectable schemes that are assigned to different operating modes. For example, a mode can be provided in which at least two of the impulse generators' impulses are output in a time sequence. In this manner an impulse sequence can be produced at the output of the generator consisting of individual pulses. Additionally or alternatively, the control device can be configured to output control impulses to at least two of the impulse generators concurrently. Then these impulse generators also deliver output impulses at their outputs concurrently, such that they are added at the output of the generator. In an adjustment of the control device output impulses can be created at the output of the generator in this manner that are higher than the other output impulses. Impulse sequences having impulses of varying impulse height and/or varying impulse time gaps can be created.

The control device can be configured to output control impulses in regular time intervals in a selected operating mode. A regular output impulse sequence at the output of the generator is then produced. For creation of specific surgical effects the control device can also be configured to output control impulses after one or more predefined time patterns in other operating modes. For example, a sequence of multiple, e.g. six, individual impulses may be followed by a longer pause after which in turn a sequence of individual impulses is output.

The impulse generators of the generator comprise at least one energy storage respectively that can be configured particularly as inductor. The impulse generator output is preferably a coil coupling with the inductor in a transformer-type manner. The inductor is at least preferably not part of a system that is able to oscillate, particularly not part of a parallel oscillation circuit or of a series oscillation circuit. The impulse generator output is preferably formed by a coil coupling with the inductor in a transformer-type manner. The impulse generator is preferably configured as flyback converter. It comprises an electronic switch that selectively connects the inductor with a voltage source in order to store energy in the coil and then separates it from the voltage source in order to provide energy at the impulse generator output as impulse with high voltage.

In a preferred embodiment of the generator the outputs of the impulse generators are connected in series and thus all of them are connected with the output of the generator. The coils forming the impulse generator output respectively are thereby preferably connected in series equidirectionally such that the impulses output from the individual impulse generators arrive at the generator output having the same polarity.

If required, it is however also possible to connect one or multiple of the coils in the opposite sense in series with the other coils in order to be able to provide positive as well as negative voltage impulses at the generator output. In this manner not only asymmetric, but also symmetric output impulse sequences can be produced at the output of the generator.

The method according to the invention is based on the production of a sequence of current impulses by means of multiple impulse generators that are connected with one another on the output side. By using multiple impulse generators that are respectively configured to only output one single output impulse upon receipt of a control impulse, the output voltage to be created as well as the provided current of the generator can be arbitrarily designed within wide limits, wherein no transient and post impulse oscillations of oscillation circuits have to be considered.

Details of the invention are apparent from embodiments that are explained in the following description with reference to a drawing comprising the following figures:

### BRIEF DESCRIPTION OF THE DRAWINGS

FIG. 1 an overview block diagram of a generator according to the invention,

FIGS. 2 and 3 embodiments of impulse generators for the generator according to FIG. 1,

FIG. 4 a simplified circuit diagram of the generator according to FIG. 1 and

FIGS. 5 to 10 control signal diagrams and output voltage diagrams of the generator according to FIGS. 1 and 4 in different operating modes.

### DETAILED DESCRIPTION

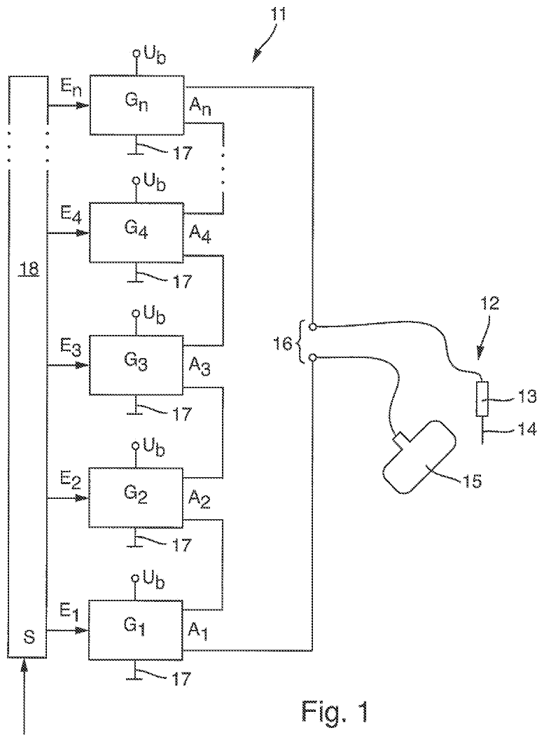

FIG. 1 illustrates a generator 11 that serves for supply of a surgical instrument 12. As an example a surgical instrument 12 for the open surgical use is illustrated in FIG. 1 having a handle 13 and an electrode 14. In this case it is a monopolar instrument. For discharging the current flowing from the instrument 12 to the patient, a neutral electrode 15 is provided that is connected via a suitable line with an output 16 of generator 11, just as the instrument 12. A monopolar instrument 12 is illustrated in FIG. 1 by way of example. Generator 11 is, however, also suitable for supply of bipolar instruments. In addition, it can also be provided for supply of instruments (probes) for laparoscopic or endoscopic use independent from whether they are monopolar or bipolar instruments. The generator 11 comprises a number of impulse generators G₁, G₂, G₃, G₄ . . . Gₙ in a suitable number, e.g. 4, 6, 12 or also in a number different therefrom. The impulse generators G₁ to Gₙ are identically configured compared with one another and are connected in parallel to each other to an operating voltage U_{b} as well as to ground 17. Basically this is not necessary; the impulse generators G₁ to Gₙ can also be configured differently in terms of their structure and/or dimensioning, e.g. in order to deliver output impulses having different amounts.

Each impulse generator G₁ to Gₙ comprises an impulse generator output A₁ to Aₙ that is respectively connected with the output 16 of generator 11. For this purpose the impulse generator outputs A₁ to Aₙ are, for example, connected in series with each other as shown in FIG. 1. This is particularly possible, because the impulse generator outputs A₁ to Aₙ are low-ohmic, i.e. they are able to forward output impulses of other impulse generators within the series connection. If the impulse generator outputs A₁ to Aₙ are high-ohmic, they can be connected in parallel to one another and can be connected with the output 16.

In addition, generator 11 comprises a control device 18 having control signal outputs that are connected with control signal inputs E₁ to Eₙ of the impulse generators G₁ to Gₙ. The control device 18 in turn comprises a control input S via which the control device 18 can receive an activation signal that can be created by means of an operating element, e.g. a foot switch, a hand switch or the like.

In figure the impulse generator G₁ is described as representative for all other impulse generators G₂ to Gₙ. The following description of impulse generator G₁ applies to all other impulse generators G₂ to Gₙ with regard to the structure of its configuration and its function accordingly.

The impulse generator G₁ is preferably configured as flyback converter. It comprises an inductor L₁, i.e., for example, a coil wound on a core having only a few windings or an air coil that is connected to the operating voltage U_{b} with one end and to an electronic switch T₁ with its other end that connects the inductor L₁ selectively with ground 17 or interrupts this connection. A protection capacitor C₁ can be assigned to the electronic switch T₁ that is connected parallel to the switchable path of the electronic switch. If the electronic switch T₁ is a transistor, e.g. a field effect transistor, the switchable path is the drain-source-path.

A control circuit V₁ is assigned to the electronic switch T₁, the control circuit V₁ being preferably effective between ground 17 and a control electrode 21 of electronic switch T₁. The control circuit V₁ can be an active or passive control circuit. It serves to open or block the controlled path of the electronic switch T₁ by means of a respective provision of signals at the control electrode 21. The control circuit V₁ comprises an input E₁ that is connected with control device 18.

The operating voltage U_{b} is provided by a DC voltage source 22, preferably via a decoupling diode 23. Diodes 23 of the individual generators G₁ to Gₙ decouple them from one another. The operating voltage U_{b} is stored in each impulse generator G₁ to Gₙ preferably on a buffer capacitor 24.

The impulse generator G₁ comprises an output A₁ that is realized by the ends of a coil 26-1 that is coupled with the inductor L₁ in a transformer-type manner. In the present embodiment inductor L₁ and coil 26-1 have equidirectional polarities. However, they could also have reverse polarities.

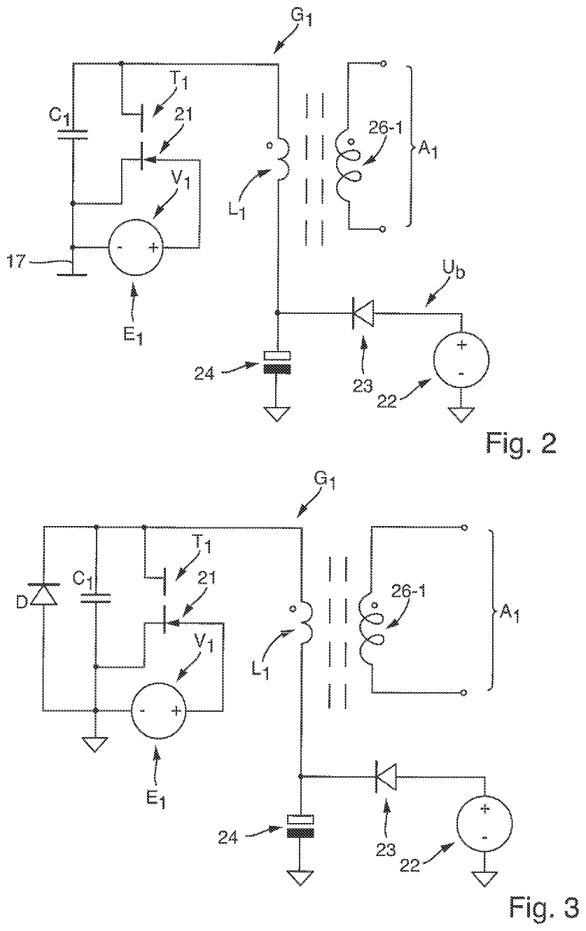

The impulse generator G₁ according to FIG. 3 is basically identical with the impulse generator G₁ according to FIG. 2. The description provided for this applies based on identical reference numerals accordingly. The particularity of impulse generator G₁ according to FIG. 3 is in the parallel connection of a diode D to the switchable path of the electronic switch T₁ in blocking direction. It protects the switch T₁ and lowers the internal resistance of impulse generator output A₁.

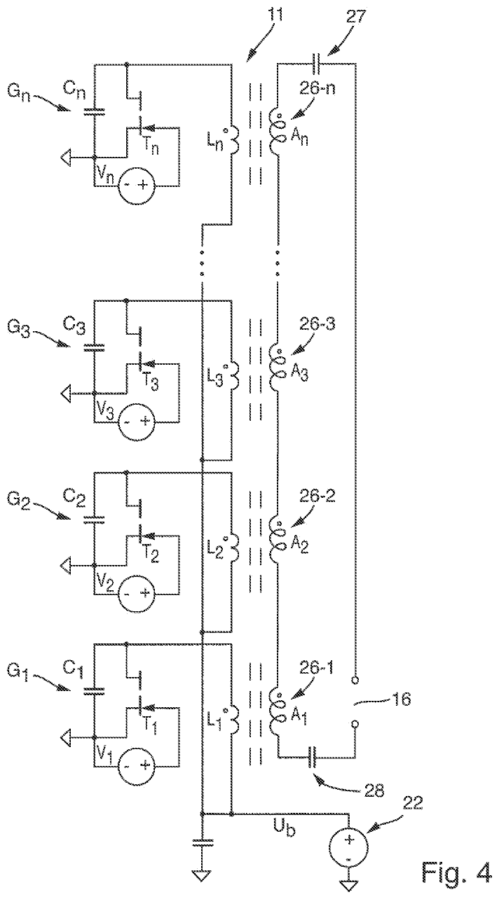

As apparent from FIG. 4, the impulse generators G₁ to Gₙ are connected in parallel with regard to the DC voltage. The switches T₁ . . . Tₙ, the inductors L₁ . . . Lₙ, the control circuits V₁ . . . Vₙ, the capacitors C₁ . . . Cₙ and the coils 26-1 to 26-n have respective letter indices that correspond to the respective impulse generators G₁ . . . Gₙ. Their impulse generator outputs A₁ . . . Aₙ, i.e. the coils 26-1 to 26-n, are, however, connected equidirectionally in series. The coil beginnings of inductors L₁ to Lₙ and of coils 26-1 to 26-n in FIG. 4 are respectively marked with a dot. The equidirectional series connection of coils 26-1 to 26-n means that a coil beginning is respectively connected with a coil end of the adjacent coil. This applies particularly, if all coil beginnings of the inductors L₁ to Lₙ are connected with identical senses, i.e. all coil beginnings are respectively connected with the electronic switch T₁ to Tₙ and all coil ends are respectively connected with the operating voltage U_{b} (or vice versa).

The series connection of impulse generator outputs A₁ . . . Aₙ can be connected with a generator output 16 via one or more coupling capacitors 27, 28. The coupling capacitors 27, 28 can also be arranged at another position of the series connection or can also be omitted alternatively. If they are provided, they eliminate the direct current component of the current output from the generator 11. If such a direct current component of the current shall be allowed, they can be omitted or can be provided with a controlled or non-controlled bridging circuit.

The generator 11 described so far operates as follows:

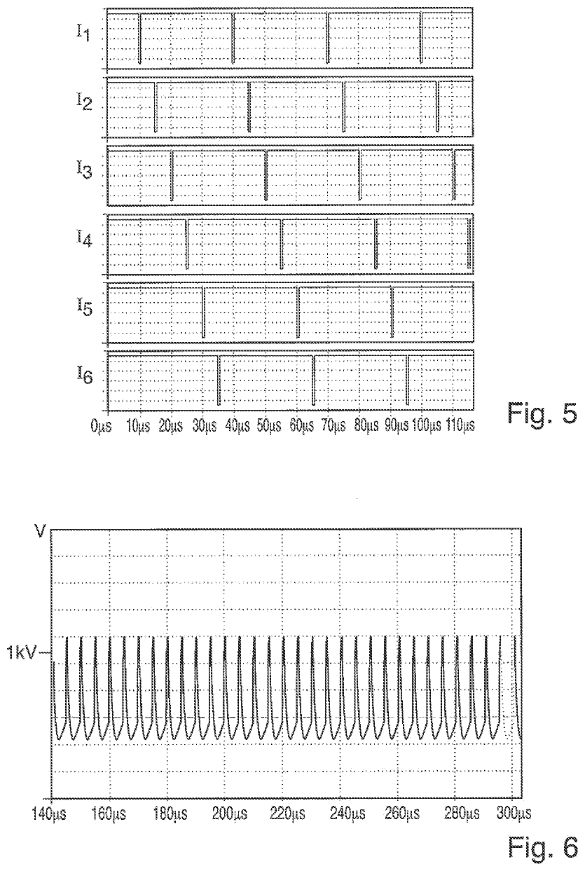

The control device 18 produces control impulses for the impulse generators G₁ to Gₙ in a timely coordinated manner. FIG. 5 illustrates such control impulses I₁ to I₆ for a generator 11 having six impulse generators G₁ to G₆ by way of example of an operating mode (first mode) having regular time intervals. As shown in FIG. 5, the control impulse I₂ for the impulse generator G₂ can be output after a predefined time period, e.g. 5 μs, after the control impulse I₁ for impulse generator G₁ has been output. This applies accordingly for the other control impulses I₃, I₄, I₅ and I₆. I₃ can be output by a time delay, e.g. 5 μs, later than I₂, I₄ later than I₃, etc. In the example according to FIG. 5, always a time interval of 5 μs is provided. The impulse generators G₁ to Gₙ thus obtain their control impulses I₁ to Iₙ in time intervals of 30 μs respectively (six impulse generators G₁ to G₆ multiplied with 5 μs). Within this period all other impulse generators G₁ to G₆ receive their control impulses I₁ . . . Iₙ in a non-varying time interval, e.g. 5 μs in the present case. Accordingly, the individual impulse generators G₁ to G₆ output their output impulses sequentially in a 5 μs-interval. The output impulse sequence schematically illustrated in FIG. 6 results at the output 16 of generator 11. The individual impulses of impulse generators G₁ to Gₙ are to a great extent of identical magnitude and together form an impulse sequence having a basic frequency of 333 kHz. The voltage provided at the output 16 is an asymmetric high frequency voltage. With other time intervals between the control impulses I₁ . . . Iₙ, also other basic frequencies can be obtained.

The control impulses I₁ . . . Iₙ are blocking impulses for the electronic switches T₁ . . . Tₙ. As control impulses, however, also all other signal shapes can be used that are suitable to cause the impulse generators G₁ . . . Gₙ to output an output impulse or also a sequence of output impulses.

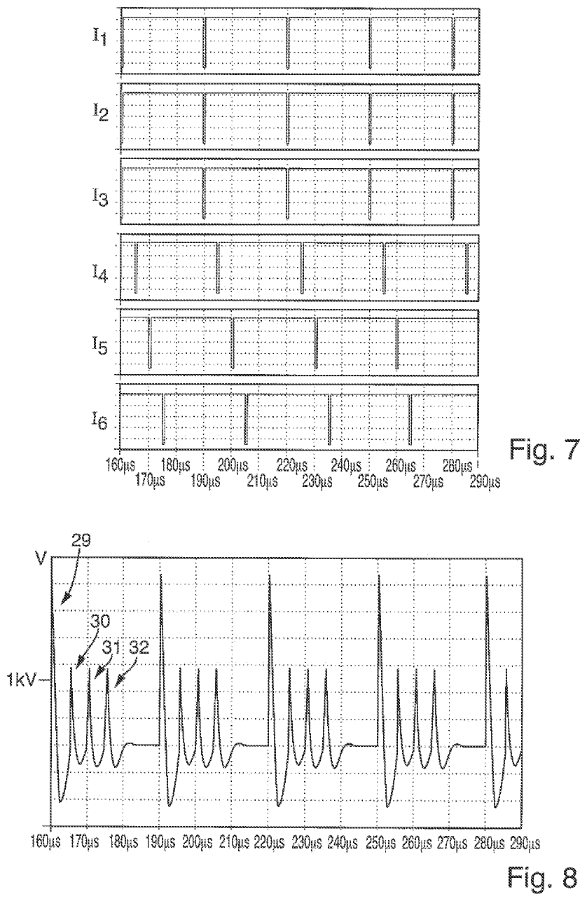

FIG. 7 illustrates another control pattern in which the control signals, i.e. the control impulses I₁, I₂, I₃, for the impulse generators G₁, G₂, G₃ are supplied to the respective impulse generators concurrently for a second operating mode (second mode), while the control impulses I₄, I₅ and I₆ are provided to the impulse generators G₄, G₅, G₆ timely sequentially. At the output 16 of generator 11 the impulse sequence, according to FIG. 8, is produced having a high first output impulse 29 that has been created by means of superimposition of output impulses of the three concurrently operating generators G₁, G₂ and G₃. The further output impulses 30, 31 and 32 are single output impulses of individual impulse generators respectively. After output of the final output impulse 32 a pause of arbitrary duration, e.g. 15 μs in the present case, can follow.

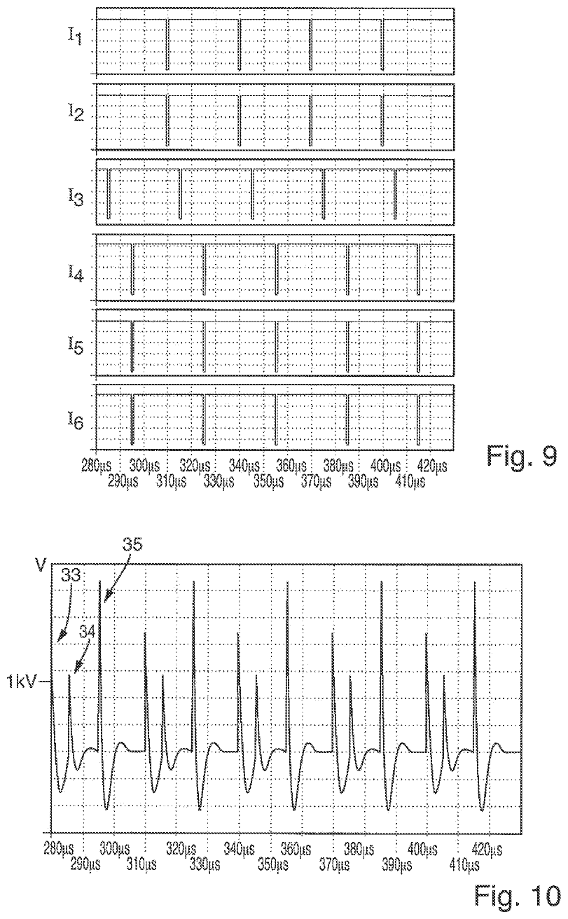

Another example for explanation of the flexibility of the signal configuration results from FIGS. 9 and 10 that illustrate a third operating mode (third mode). According to FIG. 9, generators G₁ and G₂ are synchronously provided with control impulses I₁ and I₂ and thus create doubled output impulse 33. It follows the control impulse I₃ for generator G₃ that creates a single (one fold) output impulse 34. The impulse generators G₄, G₅ and G₆ receive their control impulses I₄, I₅, I₆ in turn concurrently and thus create a triple output impulse 35.

As apparent due to the timing of control impulses I₁ to I₆, different output voltage shapes can be created that could not have been produced with a resonance generator. In doing so, the presented circuit principle provides the possibility of production of voltage shapes with physiological effects that could not have been created with previous generators. Apart from the modes illustrated explicitly here, additional modes can be produced, in that the timing of the control impulses I₁ . . . Iₙ and thus of the output impulses A₁ . . . Aₙ of impulse generators G₁ . . . Gₙ and/or the number of impulse generators G₁ . . . Gₙ and/or the plurality of the coils 26-1 . . . 26-n is varied.

A generator 11, according to the invention, comprises a number of impulse generators G₁ to Gₙ that are individually controlled by means of a control device 18 in a timely flexible manner. The RF voltage required for supply of a surgical instrument 12 is thus composed of individual impulses. The same applies for the current flowing at the electrode 14 of the instrument 12. Due to omitting resonance effects in the impulse generators G₁ to Gₙ and omitting of energy storage in a system that is able to oscillate (system of second order), the user has an increased degree of control of the wave forms of the voltage supplied to the instrument 12 and the current flowing to the instrument 12.

### LIST OF REFERENCE SIGNS

11 generator
12 instrument
13 handle
14 electrode
15 neutral electrode
16 output
G₁ . . . Gₙ impulse generators
U_{b} operating voltage
17 ground
A₁ . . . Aₙ impulse generator outputs
18 control device
L₁ . . . Lₙ inductors
E₁ . . . E₂ control signal inputs of impulse generators G₁ . . . Gₙ
I₁ . . . Iₙ control impulses for impulse generators G₁ . . . Gₙ
S control input
T₁ . . . Tₙ electronic switch
C₁ . . . Cₙ protection capacitors
V₁ . . . Vₙ control circuits
21 control electrode
22 DC voltage source
23 decoupling diode
24 buffer capacitor
26-1 . . . 26-n coils
27, 28 coupling capacitors
29 . . . 35 output impulses

1. A generator comprising:
an output configured to be connected to a medical instrument;
a plurality of impulse generators, individual ones of which comprise a control input and an impulse generator output; and
a control device that is connected with the control inputs to provide a control impulse to a respective impulse generator of the plurality of impulse generators to cause the respective impulse generator to output an output impulse, wherein the impulse generator outputs of each of the plurality of impulse generators are connected with the output of the generator.

2. The generator according to claim 1, wherein each of the plurality of impulse generators are identically configured compared with one another.

3. The generator according to claim 1, wherein each of the plurality of impulse generators is configured to output an output impulse at its impulse generator output in reaction to receipt of a control impulse at its control input.

4. The generator according to claim 1, wherein the control device is configured to output control impulses to at least two of the plurality of impulse generators sequentially.

5. The generator according to claim 1, wherein the control device is configured to output control impulses to at least two of the plurality of impulse generators concurrently.

6. The generator according to claim 1, wherein the control device is configured to output control impulses according to different time patterns.

7. The generator according to claim 1, wherein the impulse generator outputs are electrically connected in series.

8. The generator according to claim 1, wherein each of the plurality of impulse generators comprises an energy storage.

9. The generator according to claim 8, wherein the energy storage is an inductor.

10. The generator according to claim 9, wherein the impulse generator output is a coil coupling with the inductor in a transformer-type manner.

11. The generator according to claim 1, wherein individual ones of the plurality of impulse generators are flyback converters.

12. A method for producing an electrical voltage for supply of an electrosurgical instrument with a sequence of current impulses, the method comprising:
producing a sequence of current impulses by a plurality of impulse generators that are connected with one another on their output sides.

13. The method according to claim 12, further comprising causing at least two of the plurality of impulse generators to output current impulses concurrently.

14. The method according to claim 12, further comprising causing at least two of the plurality of impulse generators to output current impulses sequentially.
